(19) United States
(12) Patent Application Publication
(10) Pub. No.: US 2024/0065592 A1
(43) Pub. Date: Feb. 29, 2024

(54) Blood Culture Sample Collection System

(71) Applicant: Becton, Dickinson and Company, Franklin Lakes, NJ (US)

(72) Inventor: Jonathan Karl Burkholz, Salt Lake City, UT (US)

(21) Appl. No.: 18/237,163

(22) Filed: Aug. 23, 2023

Related U.S. Application Data
(60) Provisional application No. 63/400,507, filed on Aug. 24, 2022.

(51) Int. Cl. A61B 5/15

(52) U.S. Cl. CPC A61B 5/150992; A61B 5/15003; A61B 5/150213

(57) ABSTRACT

A blood culture sample collection system includes a container holder defining an opening and including a needle positioned within the opening, an adapter engaged with the container holder, an access connector in fluid communication with the needle of the container holder, with the access connector configured to be connected to a vascular access device, and a collection container including: a pre-advancement position; an advanced position; and a post-collection position.

### CROSS-REFERENCE TO RELATED APPLICATION

The present application claims priority to U.S. Provisional Application No. 63/400,507, entitled “Blood Culture Sample Collection System”, filed Aug. 24, 2022, the entire disclosure of which is hereby incorporated by reference in its entirety.

### BACKGROUND OF THE INVENTION

### Field of the Invention

The present disclosure relates to a blood culture sample collection system.

### Description of Related Art

When collecting a blood specimen from an indwelling vascular access devices, like a peripheral IV catheter (PIVC), a central venous catheter (CVC), or a peripherally inserted central catheter (PICC), the first 1-10 ml of blood is often wasted (i.e., disposed of) to avoid contamination from fluids in the dead space of the vascular access path of such devices. This extra step, while reducing contamination of the blood specimen, may be forgotten or improperly performed, thus compromising the blood specimen.

Blood cultures are often used as a tool to detect the presence of bacteria or fungi in a blood sample of a patient, to identify the type of bacteria or fungi present, and to direct the treatment of the patient. However, accidental contamination of the blood sample is a common problem, causing false positives and often resulting in a patient being prescribed unnecessary treatments such as, e.g., broad spectrum antibiotics. To address this concern, some healthcare providers clean the skin of the patient prior to a blood draw procedure. While this reduces the false positive rate, the rate is still significant (e.g., 3-5%) due to bacteria and/or fungi residing in, e.g., hair follicles. Therefore, some systems also divert a small volume of the initial blood drawn, with the initial (and potentially contaminated) volume being discarded. These systems, however, can be costly and time-consuming. Furthermore, these systems often rely on puncturing a patient's skin to collect the sample, which is uncomfortable for the patient.

Additionally, needle-free blood draw systems, such as PIVO™ from Becton, Dickinson and Company, are intended to be used in conjunction with intravenous catheters disposed within the patient. These needle-free blood draw systems may be configured to receive evacuated tubes such that the evacuated tubes, when in fluidic communication with a patient's vasculature system via the needle-free blood draw system, can draw blood into a reservoir of the evacuated tube due to the pressure differential between the reservoir and the patient's vasculature. Since the evacuated tubes are often not provided in a sterile condition, each time an evacuated tube is coupled to the needle-free blood draw system, there may be a risk of contamination. Even when healthcare providers disinfect the surface of a resealable membrane of an evacuated tube (e.g., with an alcohol pad), the contamination risk to the needle-free blood draw system may be too high to use the evacuated tube as a waste tube collector prior to using the needle-free blood draw system to collect a blood sample for blood culturing.

In order to address the potential for sample contamination due to contact with unsterilized evacuated tubes, blood draw systems have been developed such that the evacuated tube configured to receive a discard blood sample is held, via an adapter, in a pre-advanced position within a LLAD, which allows both the evacuated tube and the LLAD to be sterilized together, thereby reducing the number of non-sterile steps and connections during a blood culture collection procedure. Examples of such a system are shown and described in U.S. Application Publication No. 2021/0196167, which is incorporated by reference herein in its entirety. However, while the systems shown and described in U.S. Application Publication No. 2021/0196167 address at least some of the risk of a false positive blood culture sample, they do not typically account for the risk of hemolysis in subsequent blood samples collected after the initial blood culture sample is taken. A common problem with using a vascular access device such as a PIVC to draw blood from a patient is that as blood is drawn into, e.g., a blood collection tube, red blood cells are in a high shear stress state and, thus, are susceptible to hemolysis due to a high pressure differential between the vein and the blood collection tube. Such hemolysis may result in the rejection and discard of a blood sample.

### SUMMARY OF THE INVENTION

In one aspect or embodiment, a blood culture sample collection system includes a container holder having a first end and a second end positioned opposite the first end, with the container holder defining an opening and including a needle positioned within the opening, an adapter engaged with the container holder, an access connector in fluid communication with the needle of the container holder, with the access connector configured to be connected to a vascular access device, and a collection container having a first end and a second end positioned opposite the first end. The collection container has: a pre-advancement position where the first end of the collection container is spaced from the needle, the first end of the collection container is received within the opening of the container holder, and the collection container is engaged with the adapter; an advanced position where the first end of the collection container is received within the opening of the container holder, the needle is in fluid communication with the collection container, and the collection container is engaged with the adapter; and a post-collection position where the collection container is positioned outside of the opening of the container holder.

The adapter may be engaged with the collection container and separated from the container holder when the collection container is in the post-collection position. The container holder may include an elastomeric sleeve enclosing the needle. The collection container may include a neck portion and a main body, with the neck portion increasing in diameter in a direction extending from the first end of the collection container to the second end of the collection container. The collection container may include a blood culture sample collection container, where the collection container includes a septum positioned at the first end of the collection container, and where the needle pierces the septum when the collection container moves from the pre-advancement position to the advanced position. The access connector may include a luer connector. The access connector may be connected to the container holder via flexible tubing. The access connector may be compact connector with a fluid path configured to reduce a risk of hemolysis. The access connector may be directly connected to the container holder.

The adapter may be annular, with the adapter including a first portion configured to be received within the opening of the container holder and a second portion configured to engage the second end of the container holder. The second portion of the adapter may be larger in diameter than the first portion of the adapter. More than half of the neck portion of the collection container may be received within the container holder when the collection container is in the advanced position.

The blood culture sample collection system may include a vascular access device including a passive diversion portion, with the vascular access device in fluid communication with the needle. The blood culture sample collection system may include a vascular access device including an integrated catheter and a vented diversion chamber, with the vascular access device in fluid communication with the needle. The blood culture sample collection system may include a vascular access device including an extension set and a vented diversion chamber, with the vascular access device in fluid communication with the needle. The blood culture sample collection system may include a user-controlled active diversion mechanism. The blood culture sample collection system may include a blood draw device in fluid communication with the needle. The blood draw device may include a vented diversion chamber.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a front view of a blood culture sample collection system according to one aspect or embodiment of the present application;

FIG. 2 is a perspective view of an access system connector according to a further aspect or embodiment of the present application;

FIG. 3 is a front view of a blood culture sample collection system according to a further aspect or embodiment, showing a pre-advancement position of a collection container;

FIG. 4 is a front view of a blood culture sample collection system according to a further aspect or embodiment, showing an advanced position of a collection container;

FIG. 5 is a front view of a blood culture sample collection system according to a further aspect or embodiment, showing post-collection position of a collection container;

FIG. 6 is a front view of a blood culture sample collection system according to a further aspect or embodiment;

FIG. 7 is a front view of a blood culture sample collection system according to a further aspect or embodiment;

FIG. 8 is a front view of a blood culture sample collection system according to a further aspect or embodiment;

FIG. 9 is a front view of a blood culture sample collection system according to a further aspect or embodiment; and

FIG. 10 is a front view of a blood culture sample collection system according to a further aspect or embodiment.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

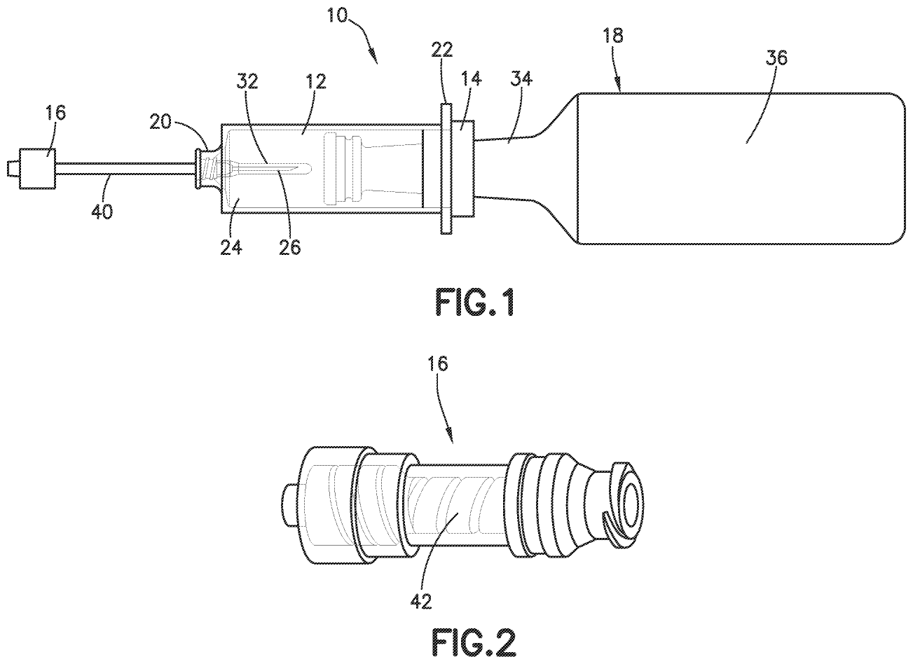

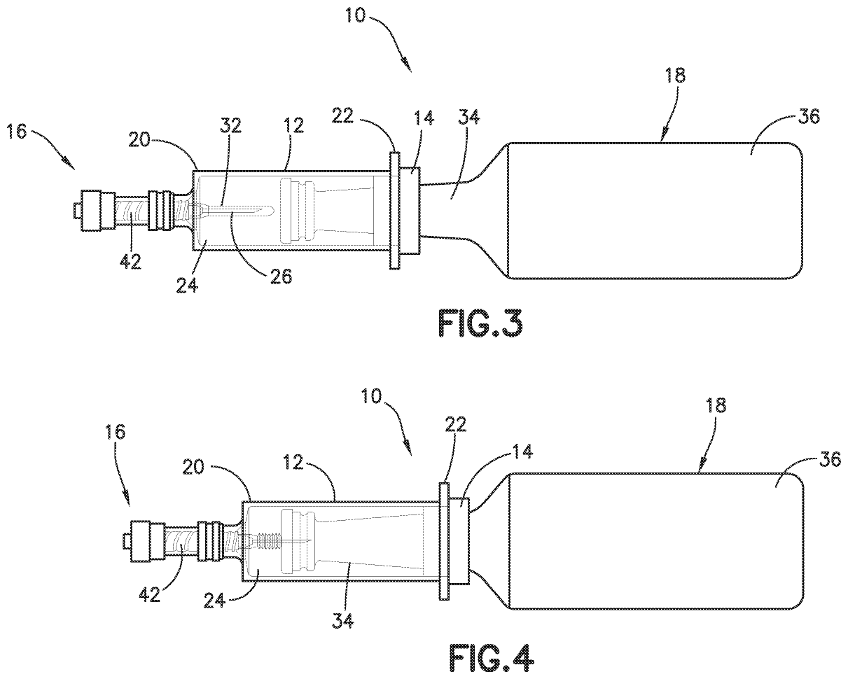

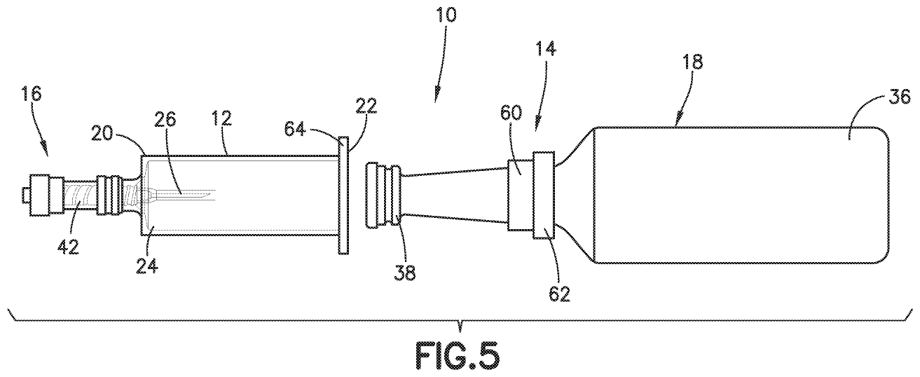

Referring to FIGS. 1-5, in one aspect or embodiment, a blood culture sample collection system 10 includes a container holder 12, an adapter 14, an access connector 16, and a collection container 18. The container holder 12 has a first end 20 and a second end 22 positioned opposite the first end 20. The container holder 12 defines an opening 24 and includes a needle 26 positioned within the opening 24. The adapter 14 is engaged with the container holder 12. In some aspects or embodiments, the adapter 14 is at least partially received by the opening 24 of the container holder 12. The access connector 16 is in fluid communication with the needle 26 of the container holder 12 and the access connector 16 is configured to be connected to a vascular access device, as discussed in more detail below. The collection container 18 has a first end 28 and a second end 30 positioned opposite the first end 28. As shown in FIGS. 3-5, the collection container 18 has a pre-advancement position (FIG. 3), an advanced position (FIG. 4), and a post-collection position (FIG. 5). In the pre-advancement position of the collection container 18, the first end 28 of the collection container 18 is spaced from the needle 26, the first end 28 of the collection container 18 is received within the opening 24 of the container holder 12, and the collection container 18 is engaged with the adapter 14. In the advanced position of the collection container 18, the first end 28 of the collection container 18 is received within the opening 24 of the container holder 12, the needle 26 is in fluid communication with the collection container 18, and the collection container 18 is engaged with the adapter 14. In the post-collection position of the collection container 18, the collection container 18 is positioned outside of the opening 24 of the container holder 12. The blood culture sample collection system 10 is configured to be assembled and sterilized with the collection container 18 in the pre-advancement position, thereby eliminating connection steps and reducing the risk of sample contamination. As shown in FIG. 5, in one aspect or embodiment, the adapter 14 is engaged with the collection container 18 and separated from the container holder 12 when the collection container 18 is in the post-collection position.

In some aspects or embodiments, the container holder 12 is an access device, such as the BD Vacutainer® Luer-Lok™ access device commercially available from Becton, Dickinson and Co. The container holder 12 includes an elastomeric sleeve 32 enclosing the needle 26, with the elastomeric sleeve 32 configured to retract when the needle 26 pierces the collection container 18 and subsequently returns to its original position to seal the needle 26. The needle 26 extends from the first end 20 of the container holder 12 into the opening 24.

In some aspects or embodiments, the collection container 18 includes a neck portion 34 and a main body 36, with the neck portion 34 increasing in diameter in a direction extending from the first end 28 of the collection container 18 to the second end 30 of the collection container 18. In one aspect or embodiment, the collection container 18 is a blood culture sample collection container, such as the BD BACTEC™ blood culture sample collection container commercially available from Becton, Dickinson and Co. The collection container 18 may include a septum 38 positioned at the first end 28 of the collection container 18, with the needle 26 piercing the septum 38 when the collection container 18 moves from the pre-advancement position to the advanced position. More than half of the neck portion 34 of the collection container 18 is received within the container holder 12 when the collection container is in the advanced position, although other suitable arrangements may be utilized.

Referring to FIG. 1, in one aspect or embodiment, the access connector 16 includes a luer connector, with the access connector 16 connected to the container holder via flexible tubing 40. The access connector 16 may be a threaded or slip luer, a threaded luer lock with a collar, a blunt plastic cannula, a male luer with clips, a cannula for PRN access, or any other suitable arrangement. The flexible tubing 40 is configured to have optimized fluidic resistance for blood collection. The flexible tubing 40 may be directly fixed or molded to the container holder 12 or may be connected to the container holder 12 via a suitable connector.

Referring to FIGS. 2-5, in one aspect or embodiment, the access connector 16 is a compact connector 42 with a fluid path configured to reduce the risk of hemolysis. The access connector 16 is directly connected to the container holder 12 via a luer connection or any other suitable connection arrangement. In some aspects or embodiments, the access connector 16 and/or flexible tubing 40 includes one of the flow path geometries shown and described in U.S. Patent Application Publication No. 2021/10186394, which is hereby incorporated by reference in its entirety. In some aspects or embodiments, the container holder 12 is directly coupled to the access connector 16 and does not include the flexible tubing 40 or the compact connector 42.

Referring again to FIGS. 3-5, in one aspect or embodiment, the adapter 14 is annular and includes a first portion 60 configured to be received within the opening 24 of the container holder 12 and a second portion 62 configured to engage the second end 22 of the container holder 12. The second portion 62 of the adapter 14 is larger in diameter than the first portion 60 of the adapter 14. The second portion 62 of the adapter 14 engages a flange 64 extending radially outward from the second end 22 of the container holder 12. The first portion 60 of the adapter is removably connected to the container holder 12 by a press or interference fit. The adapter 14 may be formed from plastic or an elastomeric material. When the collection container 18 is in the pre-advancement position, as shown in FIG. 3, the adapter 14 is configured to receive and engage the neck portion 34 of the collection container 18. In one aspect or embodiment, the adapter 14 is configured to form a seal to maintain the sterility within the opening 24 of the container holder 12 when the collection container 18 is in the pre-advancement position.

The collection container 18 is moved from the pre-advancement position to the advanced position, shown in FIG. 4, by axially moving the collection container 18 relative to the container holder 12 such that the neck portion 34 of the collection container 18 is moved further within the opening 24 of the container holder 12, with the needle 26 piercing the septum 38 and placing the needle 26 in fluid communication with the collection container 18. The collection container 18 is moved from the advanced position to the post-collection position, shown in FIG. 5, by axially moving the collection container 18 away from the container holder 12. Due to the tapering of the neck portion 34 of the collection container 18, the adapter 14 is pressed onto the neck portion 34 of the collection container 18 such that the adapter 14 is removed from the container holder 12 when the collection container 18 is moved to the post-collection position.

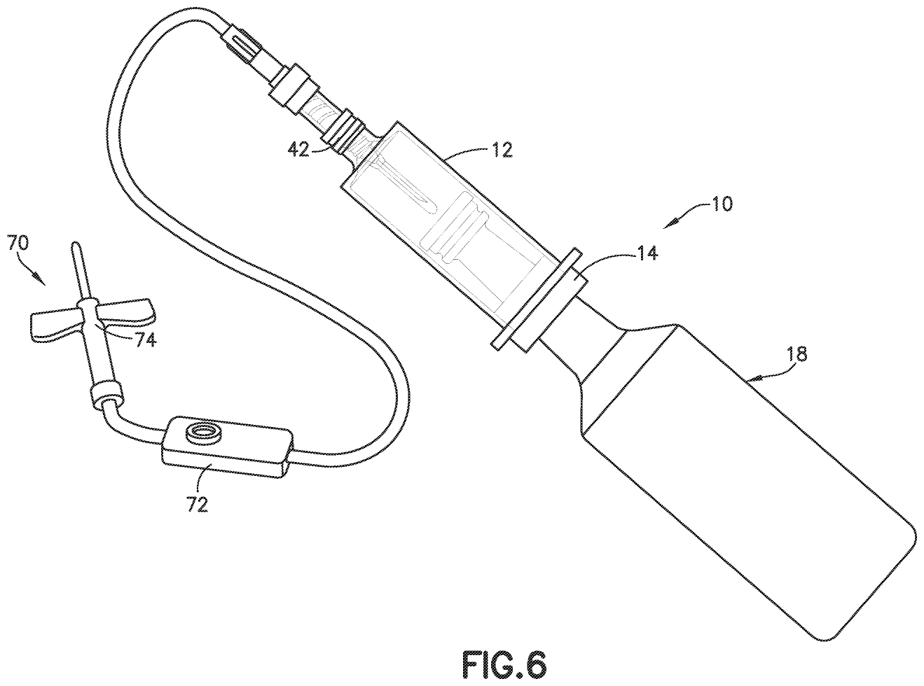

Referring to FIG. 6, in one aspect or embodiment, the blood culture sample collection system 10 further includes and is utilized with a vascular access device 70 including a passive diversion portion 72. The vascular access device 70 is in fluid communication with the needle 26. The vascular access device 70 shown in FIG. 6 is the Kurin® blood culture collection set. The passive diversion portion 72 is configured to serve as a flash chamber to provide visual confirmation of proper needle placement and capture a pre-determined volume of blood to minimize blood culture sample contamination. The vascular access device 70 includes a butterfly needle 74 to perform a standard venipuncture procedure.

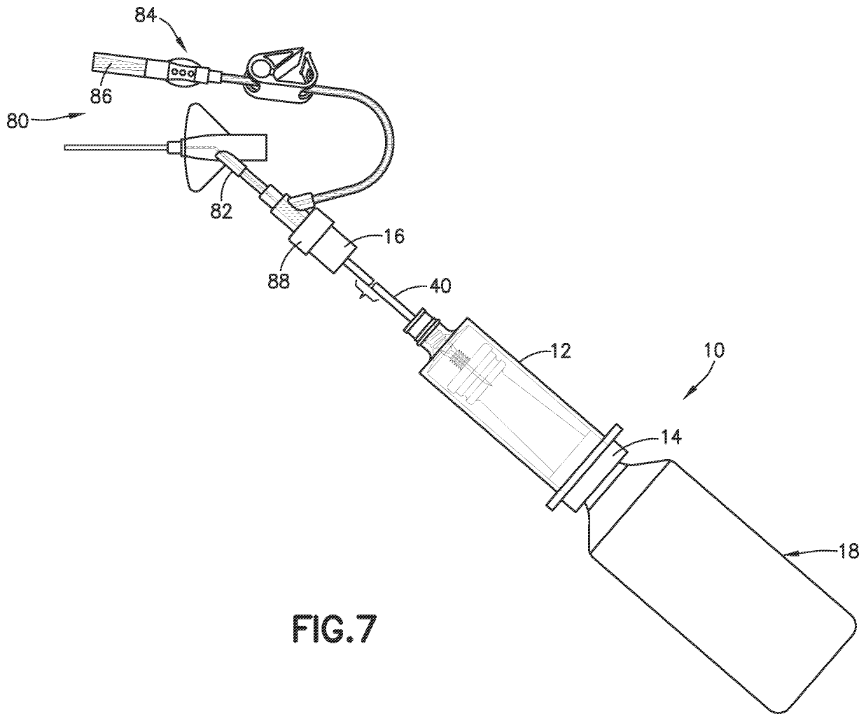

Referring to FIG. 7, in one aspect or embodiment, the blood culture sample collection system 10 further includes and is utilized with a vascular access device 80 including an integrated catheter 82 and a vented diversion chamber 84, with the vascular access device 80 in fluid communication with the needle 26. The vented diversion chamber 84 includes a vent plug 86 and is configured to passively receive a diversion volume to minimize blood culture sample contamination. The access connector 16 of the blood culture sample device 10 is connected to a needle-free connector 88 of the integrated catheter 82.

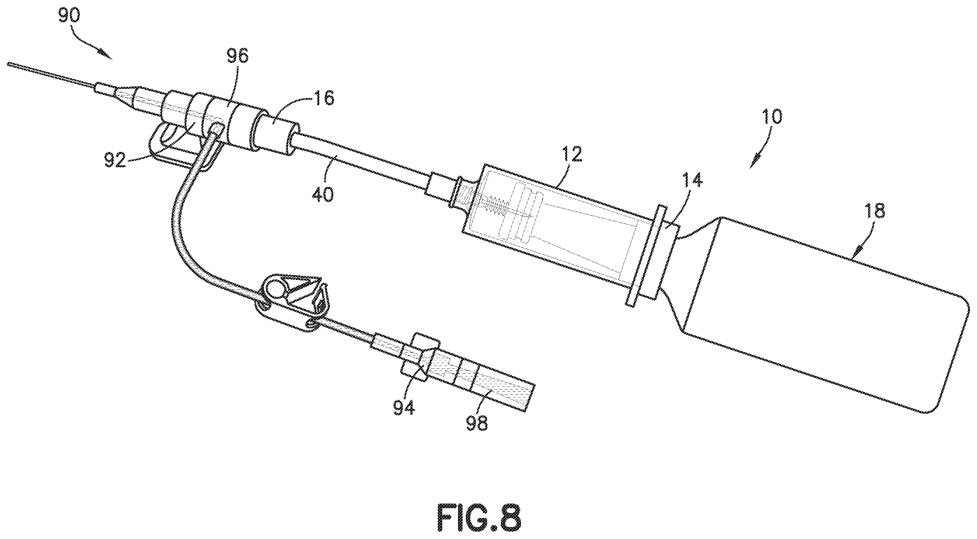

Referring to FIG. 8, in one aspect or embodiment, the blood culture sample collection system 10 further includes and is utilized with a vascular access device 90 including an extension set 92 and a vented diversion chamber 94, with the vascular access device 90 in fluid communication with the needle 26. The access connector 16 of the blood culture sample collection system 10 is connected to a distal access port 96 of the vascular access device 90. The vented diversion chamber 94 includes a vent plug 98 and is configured to passively receive a diversion volume to minimize blood culture sample contamination. The vascular access device 90 may include a conventional needle cannula or catheter.

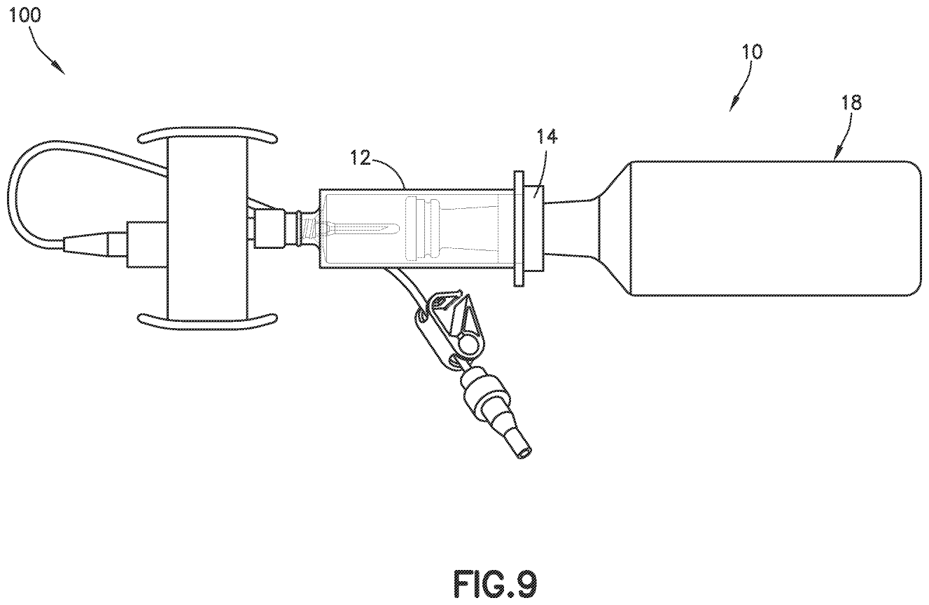

Referring to FIG. 9, in one aspect or embodiment, the blood culture sample collection system 10 further includes and is utilized with a user-controlled active diversion mechanism 100. As shown in FIG. 9, the user-controlled active diversion mechanism 100 is the Steripath® blood diversion system commercially available from Magnolia Medical Technologies. The user-controlled active diversion mechanism 100 is configured to divert and sequester a pre-determined volume of blood to minimize blood culture sample contamination.

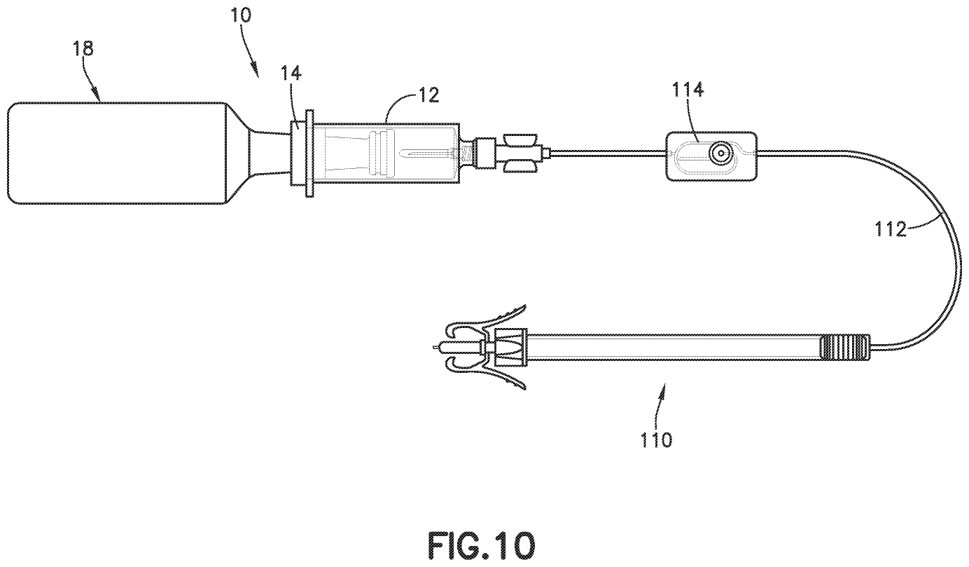

Referring to FIG. 10, in one aspect or embodiment, the blood culture sample collection system 10 further includes and is utilized with a blood draw device 110 in fluid communication with the needle 26. The blood draw device 110 may be the PIVO™ blood draw device commercially available from Velano Vascular. In one aspect or embodiment, the blood draw device 110 is the same or similar to the blood draw device shown in U.S. Pat. No. 11,090,461, which is hereby incorporated by reference in its entirety. The access connector 16 of the blood culture sample collection system 10 is connected to a connector of a fluid path 112 of the blood draw device 110. In some aspects or embodiments, the fluid path 112 includes a vented diversion chamber 114. The vented diversion chamber 114 may be automatically or manually vented after the blood draw device 110 is advanced into the vasculature of a patient.

The blood culture sample collection system 10 is configured to enable blood culture sample collection from any existing vascular access device. The blood culture sample collection system 10 is configured to reduce risk of contamination for a blood culture sample by providing all of the components of the system in sterile packaging, including the collection container. The blood culture sample collection system 10 is configured to support vascular access devices that have a diversion volume that eliminates the need for a discard sample. The blood culture sample collection system 10 is configured to reduce the number of steps to obtain a blood culture sample and improve workflow by reducing the number of connections and disconnections, thereby reducing the chances of contamination during blood draw. The blood culture sample collection system 10 is also configured to provide a system for vacuum tube blood collection immediately after blood culture sample collection that minimizes hemolysis.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A blood culture sample collection system comprising:
a container holder having a first end and a second end positioned opposite the first end, the container holder defining an opening, the container holder comprising a needle positioned within the opening;
an adapter engaged with the container holder;
an access connector in fluid communication with the needle of the container holder, the access connector configured to be connected to a vascular access device; and
a collection container having a first end and a second end positioned opposite the first end, the collection container including:
a pre-advancement position where the first end of the collection container is spaced from the needle, the first end of the collection container is received within the opening of the container holder, and the collection container is engaged with the adapter;
an advanced position where the first end of the collection container is received within the opening of the container holder, the needle is in fluid communication with the collection container, and the collection container is engaged with the adapter; and
a post-collection position where the collection container is positioned outside of the opening of the container holder.

2. The system of claim 1, wherein the adapter is engaged with the collection container and separated from the container holder when the collection container is in the post-collection position.

3. The system of claim 1, wherein the container holder comprises an elastomeric sleeve enclosing the needle.

4. The system of claim 1, wherein the collection container comprises a neck portion and a main body, the neck portion increasing in diameter in a direction extending from the first end of the collection container to the second end of the collection container.

5. The system of claim 1, wherein the collection container comprises a blood culture sample collection container, wherein the collection container comprises a septum positioned at the first end of the collection container, and wherein the needle pierces the septum when the collection container moves from the pre-advancement position to the advanced position.

6. The system of claim 1, wherein the access connector comprises a luer connector.

7. The system of claim 6, wherein the access connector is connected to the container holder via flexible tubing.

8. The system of claim 1, wherein the access connector comprises a compact connector with a fluid path configured to reduce a risk of hemolysis.

9. The system of claim 8, wherein the access connector is directly connected to the container holder.

10. The system of claim 1, wherein the adapter is annular, the adapter including a first portion configured to be received within the opening of the container holder and a second portion configured to engage the second end of the container holder.

11. The system of claim 10, wherein the second portion of the adapter is larger in diameter than the first portion of the adapter.

12. The system of claim 4, wherein more than half of the neck portion of the collection container is received within the container holder when the collection container is in the advanced position.

13. The system of claim 1, further comprising a vascular access device including a passive diversion portion, the vascular access device in fluid communication with the needle.

14. The system of claim 1, further comprising a vascular access device including an integrated catheter and a vented diversion chamber, the vascular access device in fluid communication with the needle.

15. The system of claim 1, further comprising a vascular access device including an extension set and a vented diversion chamber, the vascular access device in fluid communication with the needle.

16. The system of claim 1, further comprising a user-controlled active diversion mechanism.

17. The system of claim 1, further comprising a blood draw device in fluid communication with the needle.

18. The system of claim 17, further comprising a vented diversion chamber.
